(19) United States
(12) Patent Application Publication
(10) Pub. No.: US 2010/0041912 A1
(43) Pub. Date: Feb. 18, 2010

(54) METHOD FOR THE WITTIG REACTION IN THE PREPARATION OF CARBOPROST

(75) Inventors: Keshav Shripad Bhide, Bangalore (IN); Govindrao Abhijit Padhye, Bangalore (IN); Madhukar Niranjan Paingankar, Bangalore (IN)

Correspondence Address: ASTRAZENECA R&D BOSTON, 35 GATEHOUSE DRIVE, WALTHAM, MA, 02451-1215, US

(73) Assignee: Astra Zeneca, Sodertalje (SE)

(21) Appl. No.: 12/521,821

(22) PCT Filed: Jan. 5, 2007
(86) PCT No.: PCT/GB2008/000020

§ 371 Date: Oct. 7, 2009

(30) Foreign Application Priority Data
Jan. 5, 2007 (IN) 29/CHE/2007

(51) Int. Cl. C07C 69/76

(52) U.S. Cl. 560/61

(57) ABSTRACT

A process for the preparation of carboprost methyl ester (FIG. (10)).

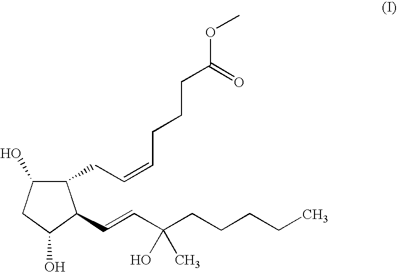

Prostaglandins are a family of 20-carbon fatty acids found in virtually all mammalian cells¹. They are highly biologically active. Natural prostaglandins possess an allylic, secondary alcohol group at C-15. This group can be oxidized into a ketone

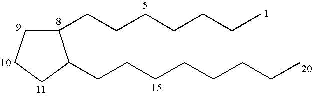

in the presence of 15-hydroxyprostaglandin dehydrogenase and this process is very rapid in vivo in animals and man. The oxidation to 15-ketoprostaglandins leads to the inactivation of prostaglandin in vivo.

Thus a number of prostaglandins with different substitutions at C-15 were synthesized in order to maintain biological activity. The first compound of this type was 15(S)-15-methyl-prostaglandin F_{2α} synthesized by Upjohn chemists (Ernest W. Yankee, Udo Axen and Gordon L. Bundy: Journal of the American Chemical Society/96:18/Sep. 4, 1974). 15(S)-15-methyl-prostaglandin F_{2α} as its tromethamine salt is used for post partum haemorrhage indication.

The method described by Yankee et al starts with the Grignard reaction of benzoate-protected enone (2). This benzoate enone (2) was synthesized from optically pure iodo lactone (1) analogous to a known literature process².

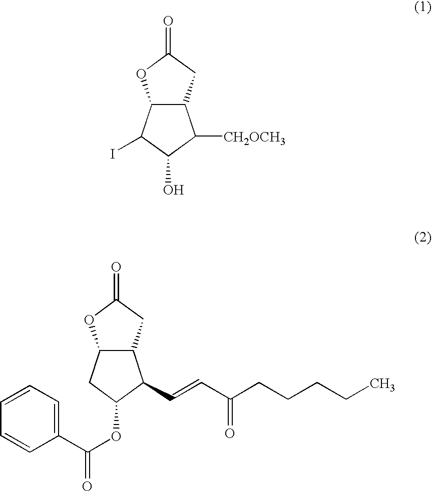

Benzoate enone (2) was treated with methyl magnesium bromide at −78° C. in ether or tetrahydrofuran as solvent or with trimethyl aluminium in benzene at ambient temperature to give 3(RS) with an epimeric ratio of 1:1.

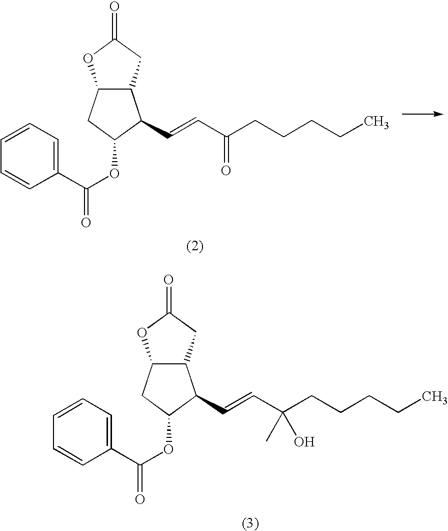

The reduction of 3 (RS) with diisobutylaluminium hydride gave a lactol product 4(RS).

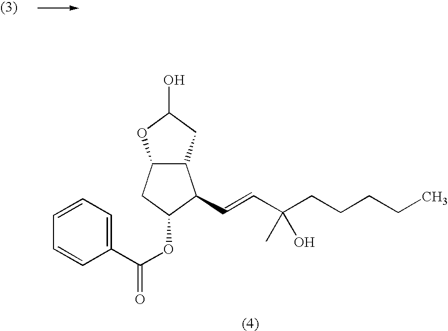

Alternatively 3 was subjected to hydrolysis using sodium methoxide in methanol to give 5 (RS), this 5 is then protected as trimethyl silyl ether using tri methylsilyldiethylamine to yield a trimethyl silyl protected ether 6 (RS).

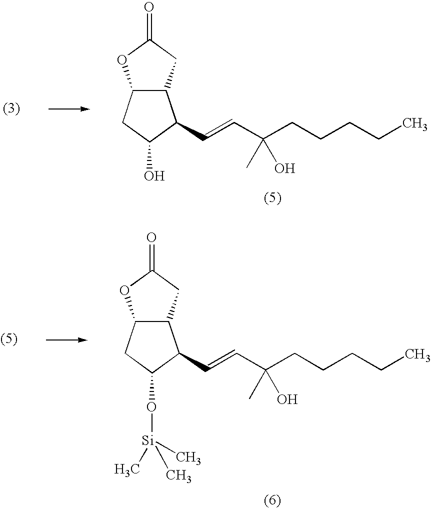

Diisobutylaluminium hydride reduction of 5 and 6 gave lactol product 7(RS) and 8 (RS) respectively.

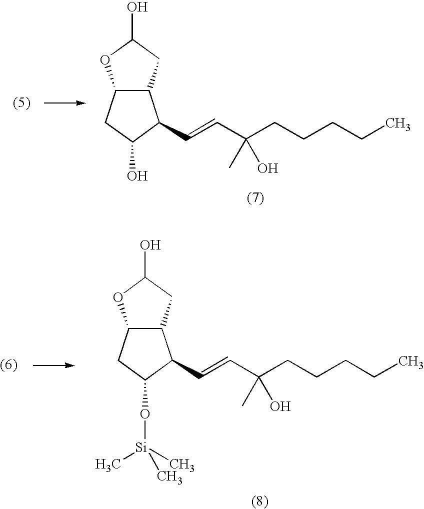

The reaction of each of these lactols i.e. 4(RS), 7(RS) and 8(RS) with the ylide prepared from 4-carboxybutyltriphenylphosphonium bromide and sodium methylsulfinylmethide gave prostaglandin 9(RS). Esterification of 9(RS) with diazomethane gave Methyl (Z)-7-(1R,2R,3R,5S)-3,5-dihydroxy-2-[(E)-(3RS)-3-hydroxy-3-methyl-1-octenyl)cyclopentyl]-5-heptenoate i.e. carboprost methyl ester 10(RS) in 28-37% overall yield starting from benzoate enone (2). The two epimers 15R and 15S are separated by column chromatography using silica gel as stationary phase and methylene chloride: acetone as the mobile phase.

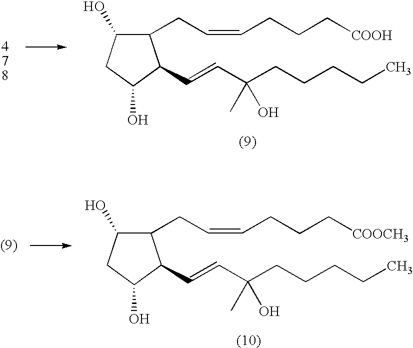

We have now devised improved processes for the production of carboprost methyl ester.

In a first aspect of the invention we provide a process for the preparation of carboprost methyl ester 10

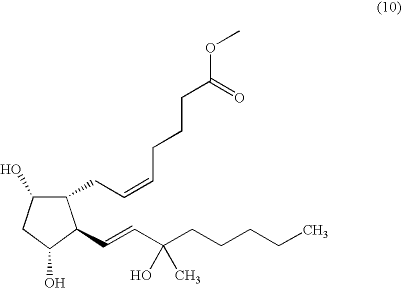

which process comprises the Wittig reaction of lactol 13

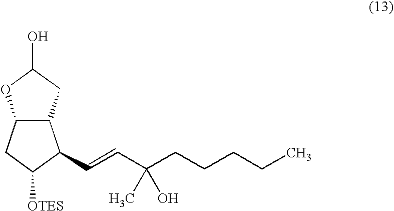

wherein OTES is a triethylsilyloxy group
with an ylide of formula Ph₃P═CH—(CH₂)₃—COOH at between −25° C. and +10° C. to give carboprost 9

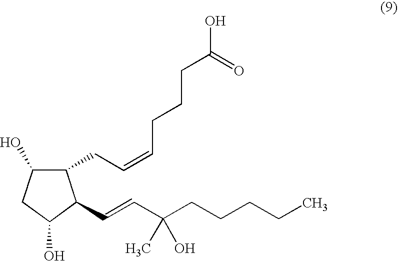

followed by esterification to give carboprost methyl ester 10.

It is to be understood that reference above to carboprost methyl ester (10), lactol (13) and carboprost (9) relates in each case to the racemate (RS) and/or each individual R or S isomer. That is to say the process may be used to prepare the racemate of (10) and/or the individual R or S isomer, or any combination thereof. The separation of the racemate may occur at any stage of the process, for example by separation of compound 13, of compound 9 or compound 10 (each selected independently) into individual R and S isomers.

Any convenient method may be used to separate a racemic mixture, a particular method is preparative scale HPLC. Convenient HPLC methods are as set out in reference 3 incorporated herein.

For the avoidance of doubt the method of the invention may be performed using either the R or S isomer of lactol 13 (each selected independently).

Yankee et al (op cit) conduct the Wittig reaction at ambient temperature (20° C. or more). We have found that this results in formation of about 6-8% of the unwanted trans-isomer. In contrast we have found that use of a lower temperature i.e. between −25° C. and +10° C. results in less than 3% of the unwanted trans-isomer. The Wittig reaction is more conveniently carried out at between −5° C. and +5° C.

The ylide of formula Ph₃P═CH—(CH₂)₃—COOH is conveniently formed by the reaction of 4-carboxybutyltriphenyl phosphonium bromide and sodium methylsulfinylmethide. In turn sodium methylsulfinylmethide is conveniently obtained by the reaction of sodium hydride with dimethylsulphoxide.

In a further aspect of the invention we have found that sodium methylsulfinylmethide can be prepared using sodium amide in place of sodium hydride. There are a number of safety hazards associated with the use of sodium hydride; in contrast sodium amide is relatively easy to handle on a large scale.

The esterification is conveniently effected using dimethyl sulphate and potassium carbonate or methyl iodide and potassium carbonate, more conveniently using methyl iodide and potassium carbonate to yield carboprost methyl ester 10. This contrasts with the method of Yankee et. al who used ethereal diazomethane for esterification. Use of ethereal diazomethane on large scale is cumbersome and also poses a major safety hazard.

Convenient solvents used in the esterification include acetone.

In a further aspect of the invention where the process is used to prepare carboprost methyl ester 10RS this is conveniently further separated into individual carboprost methyl esters 10a and 10b

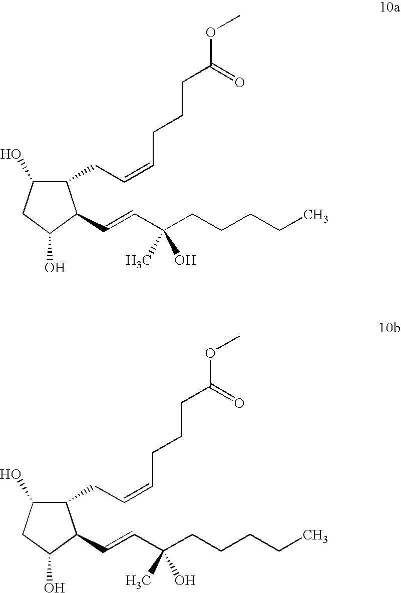

for example using preparative scale HPLC. Convenient HPLC methods are as set out in reference 3 incorporated herein by reference.

In a further aspect of the invention the lactol 13 is conveniently prepared by reduction of (3aR,4R,5R,6aS)-5-triethylsilyloxy-4-[(1E,3S)-3-hydroxy-3-methyl oct-1-en-1-yl]hexahydro-2H-cyclopenta[b]furan-2-one (12)

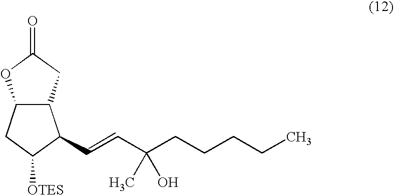

dissolved in a convenient solvent such as tetrahydrofuran, toluene or xylene, preferably tetrahydrofuran, using diisobutylaluminium hydride (1.5M solution in toluene) at between −60° C. and −78° C. We have found that about 3.5 moles of diisobutylaluminium hydride and about one molar equivalent of product 12 may be used for the reduction of 12 to 13 as compared to 4.6 moles to 5.4 moles of diisobutylaluminium hydride as described by Yankee et al. (op cit).

(3aR,4R,5R,6aS)-5-triethylsilyloxy-4-[(1E,3S)-3-hydroxy-3-methyl oct-1-en-1-yl]hexahydro-2H-cyclopenta[b]furan-2-one (12) is conveniently prepared by Grignard reaction of, for example methyl magnesium chloride with (3aR,4R,5R,6aS)-5-triethylsilyloxy-4-[(1E)-3-oxooct-1-en-1-yl]hexahydro-2H-cyclopenta[b]furan-2-one (11) (triethylsilyloxy PG enone) that had been dissolved in a convenient solvent such as tetrahydrofuran, and cooled at between −60° C. and −78° C. We have found that the ratio of compound 11 to Grignard reagent used can be about 1:5 moles. We have found that this ratio is required for the reaction to go to completion. A slight reduction in the molar quantity of Grignard reagent eg. a molar ratio of 1:4 does not take the reaction to completion. We have carried out this reaction with higher ratio of Grignard reagent and observed that higher quantity of Grignard reagent added does not play any significant role, on the contrary it may increases the impurity formation. This all contrasts with the method of Yankee et al. (op cit) who used a ratio of 1:16 moles.

In a further aspect of the invention (3aR,4R,5R,6aS)-5-triethylsilyloxy-4-[(1E,3S)-3-hydroxy-3-methyl oct-1-en-1-yl]hexahydro-2H-cyclopenta[b]furan-2-one (12) is prepared as shown in Scheme 11 hereinafter by Grignard reaction with compound 15. In turn compound 15 is prepared by protection of the hydroxyl group of compound 14 with triethyl silyl chloride (triethylchlorosilane) in for example pyridine or triethyl amine to yield compound 15 (in 95-98% yield.). Reaction of Grignard reagent (pentyl magnesium bromide, 2.0M solution in diethyl ether) with compound 15 gave compound 12RS, the structure of which was confirmed by spectroscopic data. In this case the epimeric ratio of R:S isomer obtained was 50:50.

In a further aspect of the invention compound 12 is obtained as an isomeric mixture and then separated into individual isomers 12a and 12b.

The invention will now be illustrated but not limited by reference to the following specific description and Examples.

### EXAMPLE 1

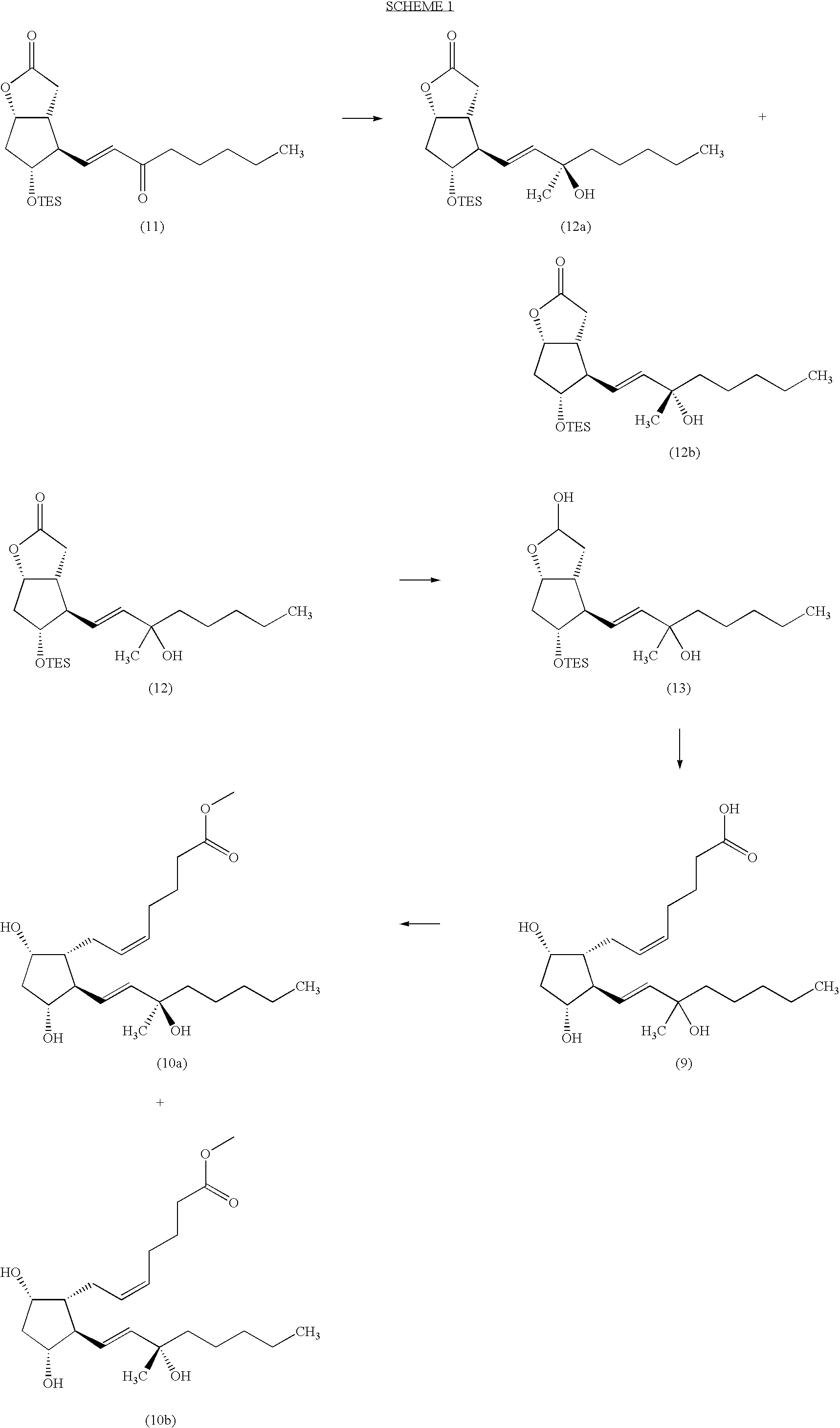

Methyl magnesium chloride (Grignard Reagent) as 3.0M solution in tetrahydrofuran was added to (3aR,4R,5R,6aS)-5-triethylsilyloxy-4-[(1E)-3-oxooct-1-en-1-yl]hexahydro-2H-cyclopenta[b]furan-2-one (11) (triethylsilyloxy PG enone) that had been dissolved in tetrahydrofuran and cooled to −78° C. The ratio of 11 to Grignard reagent used was 1:5 moles, as compared to Yankee et al. where the authors used a ratio of 1:16 moles.

The crude product was obtained as RS mixture (12a, 12b). The HPLC analysis of (12) using a Supelcosil silica column and mobile phase as Heptane:Isopropyl alcohol (94:6) at 200 nm absorbance revealed two peaks due to two epimers 12a and 12b in a ratio of 40:60 and a bunch of impurities formed to an extent of 30% (by area %). These impurities were not characterized. The R:S ratio was assigned to be 40:60 that was unambiguously confirmed, the details of this are discussed in relation to Scheme III hereinafter.

Based on the HPLC data we decided to look into the possibility of reducing the impurities and consequently increase the yield. HPLC data revealed an impurity formation at an retention time of 7.5 to 8.5 to an extent of 30% (by area) so it was clear that if we were able to control or bring down this impurity, the yield of 12(RS) would subsequently increase. We also wanted to investigate increasing the percentage of S isomer.

Analyzing the various parameters it appeared that solvent was playing a major role. Various non-polar solvents like heptane, pentane, hexane, xylene (o, m, p or mixed) and toluene were used to dissolve the substrate i.e. triethylsilyloxy PG enone (11). Methyl magnesium chloride (Grignard Reagent) as 3.0M solution in tetrahydrofuran was added at −78° C. to the solution of 11 which was dissolved in heptane, pentane, hexane, xylene (o, m, p or mixed) or toluene. The ratio of 11 to Grignard reagent used is 1:5 moles. In solvents like hexane, pentane and heptane the reaction did not go to completion using, 5.0 moles of Grignard reagent. However, in solvents like xylene (o, m, p or mixed) and toluene the reaction proceeded to completion. The HPLC analysis [Supelcosil silica column and mobile phase as Heptane:Isopropyl alcohol (94:6) at 200 nm] of (12) obtained as a RS mixture by changing the solvent revealed that the two epimers 12a and 12b were formed in a ratio of 30:70 and the impurities were formed only to an extent of 4-6% (by area %).

The crude product 12 as a RS mixture (12a & 12b) dissolved in tetrahydrofuran was reduced to lactol 13 (RS) using diisobutylaluminium hydride (1.5M solution in toluene) at −78° C. We used 3.5 moles of diisobutylaluminium hydride for the reduction of 12 to 13(RS) [as compared to 4.6 moles to 5.4 moles of diisobutylaluminium hydride used for the reduction of 12 as described by Yankee et al.]

Treatment of the lactol 13 (RS) with the ylide prepared from 4-carboxybutyltriphenyl-phosphonium bromide and sodium methylsulfinylmethide (obtained by reaction of sodium hydride and dimethyl sulphoxide) at −5° C. to 2° C. gave 9(RS). Tetrahydrofuran, Toluene or Xylene (o, m, p or mixed) was used to dissolve the lactol product. The 5-trans isomer was formed to an extent of less than 3.0%. Yankee et al describe the addition of lactol 13(RS) to ylide at ambient temperature; our observation is that when the lactol is added to the ylide at ambient temperature the amount of 5-trans isomer obtained was around 6.0 to 8.0%. The Pharmacopoeia states the limits of 5-trans isomer to be not more than 4.0%.

Yankee et al have reported the synthesis of sodium methylsulfinylmethide, which is then reacted with 4-carboxybutyltriphenyl-phosphonium bromide to give an ylide. The sodium methylsulfinylmethide is obtained by the reaction of sodium hydride and dimethyl sulphoxide. However handling of sodium hydride on large scale poses a major safety hazard. Sodium hydride is usually available as a 55-60% suspension in oil. Sodium hydride (as a suspension in oil) is slurried with petroleum ether (60-80) to remove the oil suspension. On a large scale the petroleum ether used to free the sodium hydride from the oil suspension has to be siphoned prior to the reaction, which is very cumbersome. We used sodium amide in place of sodium hydride. Sodium amide is easy to handle on large scale. Treatment of the lactol 13(RS) at −25° C. to 10° C. preferably at −5° C. to 5° C. with the ylide prepared from 4-carboxybutyltriphenyl-phosphonium bromide, sodium amide and dimethyl sulphoxide gave 9(RS) with the formation of 5-trans isomer below 3.0%.

The cleavage of the triethyl silyl group occurred during the product isolation (work up).

The carboprost acid 9(RS) obtained after Wittig reaction is subjected to esterification using dimethyl sulphate and potassium carbonate or methyl iodide and potassium carbonate preferably methyl iodide and potassium carbonate to yield 10(RS). These contrasts with that reported by Yankee et. al who used ethereal diazomethane for esterification. Use of ethereal diazomethane on large scale is cumbersome and also poses a major safety hazard.

Thus, 15-Methyl-PGF₂α Methyl ester [(Carboprost methyl ester) 10(RS)] was synthesized as shown in Scheme I in two ways. The first method started with the Grignard reaction of triethyl silyloxy PG enone (11) and methyl magnesium chloride in THF as solvent at −78° C. The Grignard product (12) thus obtained is reduced with diisobutyl aluminium hydride to yield (13), which is then subjected to Wittig reaction by reacting the ylide obtained from 4-carboxybutyltriphenyl-phosphonium bromide and sodium methylsulfinylmethide (obtained by reaction of sodium hydride and dimethyl sulphoxide) and the lactol (13) at −25° C. to 10° C., preferably at −5° C. to 5° C. to yield 9(RS). Esterification of 9(RS) using methyl iodide and potassium carbonate in acetone as solvent gave 10 (RS) in 55% overall yield starting from Triethyl silyloxy PG enone (11) with an R:S ratio of 40:60.

The second method describes the Grignard reaction of triethyl silyloxy PG enone (11) with the Grignard reagent in xylene (o, m, p or mixed) or toluene as solvent at −78° C. to yield 12(RS). The Grignard product (12) thus obtained is reduced with diisobutyl aluminium hydride to yield (13), which is then subjected to Wittig reaction by reacting the ylide obtained from 4-carboxybutyltriphenyl-phosphonium bromide and sodium methylsulfinylmethide (obtained by reaction of Sodium amide and dimethyl sulphoxide) with the lactol (13) at −25° C. to 10° C. preferably at −5° C. to 5° C. to yield 9(RS). Esterification of 9(RS) using methyl iodide and potassium carbonate in acetone as solvent gave 10 (RS) in 75% overall yield starting from triethyl silyl PG enone (11) with an R:S ratio of 30:70.

In contrast Yankee et al separated the 15-Methyl-PGF₂α Methyl ester obtained as a RS mixture into individual R and S isomer on silica column using methylene chloride: acetone as mobile phase. Column chromatography of the RS mixture gave various fractions containing either pure R isomer or mixture of RS isomers and pure S isomer. To further separate the RS mixture again into R isomer and S isomer completely one has to carry out column chromatography repeatedly. As evident on large-scale synthesis this is not feasible. Also routine column chromatography using silica stationary phase it was not possible to isolate the S trans isomer.

The invention describes the use of preparative scale HPLC for the separation of R and S isomers. Once the initial purification is done on silica column, the fractions obtained as RS mixture is taken up for separation on a preparative column using a preparative HPLC. The invention describes normal phase and a reverse phase method to separate the isomers. In the normal phase the column was packed with Chiralpak AD material supplied from Diacel, Japan. Mobile phase used was either Heptane:Ethanol, Heptane:Isopropyl alcohol, Hexane:Ethanol and Hexane:isopropyl alcohol. Heptane:Ethanol or Heptane:Isopropyl alcohol gave the best results. The ratios ranging from anywhere between 80:20 to 95:5 were used but preferably 90:10. UV detection is 200-220 nm, preferably 216 nm. In the reverse phase method Inertsil Prep ODS 50 mm (I.D) column (mobile phase water/methanol/acetonitrile) or YMC C8 column (mobile phase water/methanol) or Kromasil C18 column (mobile phase water/methanol/acetonitrile) or Merck Lichroprep (mobile phase water/methanol) was used. The best results were however obtained with Inertsil Prep ODS 50 mm (I.D) column the mobile phase was water:methanol:acetonitrile with ratios ranging from 50:15:35 to 70:15:15. However best results were obtained with a ratio of water:methanol:acetonitrile as 60:05:35 at an absorbance 200-220 nm preferably 200 nm. Using Prep HPLC the R and S isomer were efficiently separated. Also the 15(S)-5-trans isomer which otherwise was never isolated before in pure form was also isolated and characterised.

The methods of this invention for the synthesis of 15-Methyl-PGF₂α Methyl ester 10(RS) as disclosed above may include one or more of the following features:
1. The method provides a feasible, production scale synthesis of 15-Methyl-PGF₂α Methyl ester 10(RS).
2. Improved ratio of R:S isomer of 15-Methyl-PGF₂α Methyl ester (10) can be obtained 30:70.
3. The isomeric purification is efficient which in turn leads to higher yields of (15S)-15-Methy-PGF₂α Methyl ester i.e. Carboprost methyl ester.
4. The impurity (other side products) formation is only to the extent of 4-6%.
5. The use of sodium amide during Wittig reaction and/or use of methyl iodide during esterification step, thus making it a safer production scale synthesis of 15-Methyl-PGF₂α Methyl ester 10(RS).
6. The quantity of reagents i.e. Grignard reagent and/or Diisobutyl aluminium hydride used is less than used in the prior art leading to a more complete consumption of starting material(s).

The disadvantages of the method for the synthesis of 15-Methyl-PGF₂α Methyl ester (10) described in the prior art by Ernest W. Yankee, Udo Axen and Gordon L. Bundy: Journal of the American Chemical Society/96:18/Sep. 4, 1974 include:
1. The inherent drawbacks do not readily make it a feasible method for industrial scale synthesis.
2. Use of reagents like of Sodium hydride or diazomethane does not make it a safe process on industrial scale.
3. Large excess of reagents like Grignard reagent or Diisobutyl aluminium hydride is used in the reaction.
4. The efficiency of isomers purification is very low.
5. The ratio of R:S isomer of 15-Methyl-PGF₂α Methyl ester (10) obtained is 50:50, thus leading to lower yields of (15S)-15-Methyl-PGF₂α Methyl ester i.e. Carboprost methyl ester.

### EXAMPLE 2

The invention further describes a novel route (Scheme II) for the synthesis of the intermediate 12.

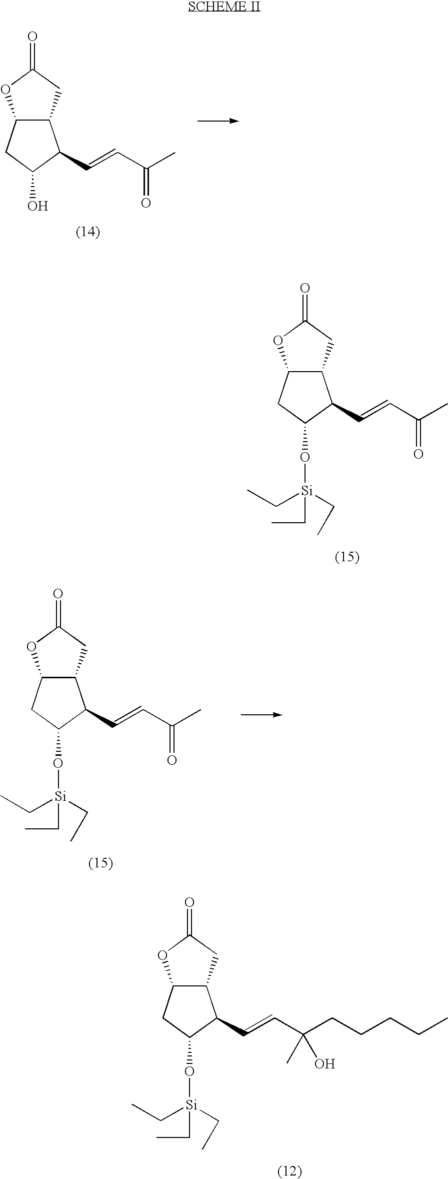

The scheme II starts with the protection of the hydroxyl group of compound 14 with triethyl silyl chloride (triethylchlorosilane) in pyridine or triethyl amine to yield compound 15 in 95-98% yield. Reaction of Grignard reagent (pentyl magnesium bromide, 2.0M solution in diethyl ether) with compound 15 gave compound 12(RS), the structure of which was confirmed by spectroscopic data. In this case the epimeric ratio of R:S isomer obtained was 50:50.

### EXAMPLE 3

The invention further describes a new approach (Scheme III) to the synthesis of either (15R)-15-Methyl-PGF₂α Methyl ester (10a) or (15S)-15-Methyl-PGF₂α Methyl ester i.e. Carboprost methyl ester (10b).

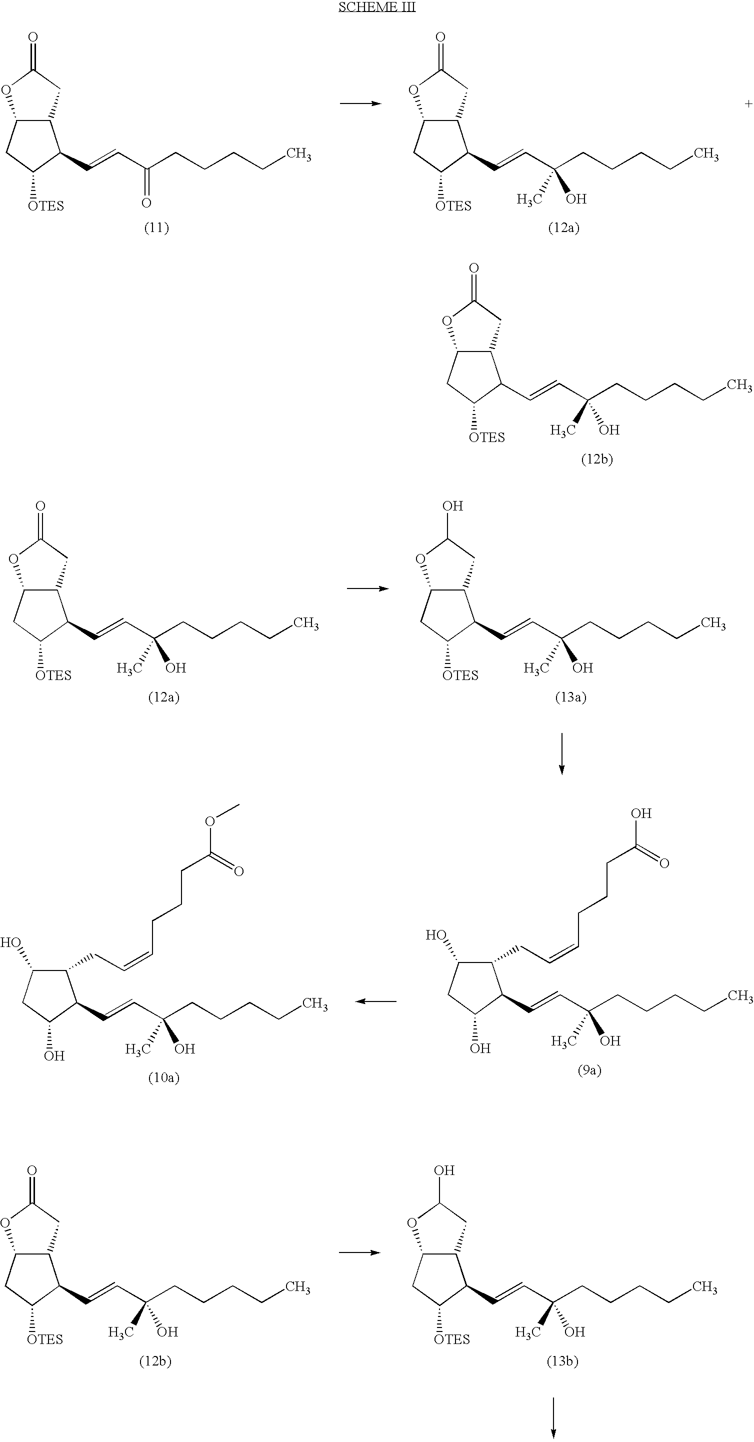

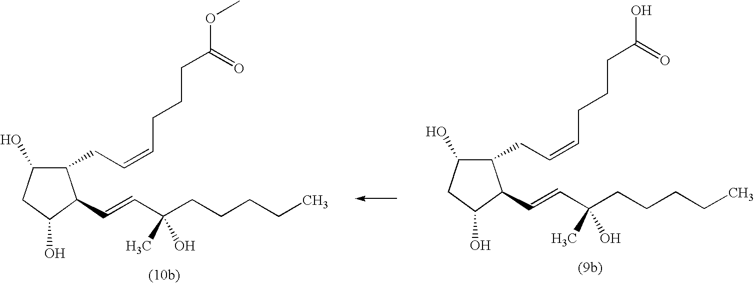

The Grignard product (12) is obtained as an isomeric (RS) mixture i.e. 12a and 12b respectively by the reaction of triethyl silyl protected PG enone (11) with the Grignard reagent (Methyl magnesium chloride). In this approach the invention describes the isomers purification at this stage rather than separating the isomers (10a and 10b) at the end. Product 12 obtained as a RS mixture is separated into individual isomers i.e. 12a and 12b respectively using preparative HPLC. Herein we describe two methods a normal phase and a reverse phase to separate the isomers 12a and 12b respectively. In the normal phase, the column was packed with Chiralpak AD material supplied from Diacel, Japan. Mobile phase used was Heptane:Isopropyl alcohol or Heptane:ethanol. The ratio ranging anywhere between 80:20 was used at absorbance 200-220 nm. However the best result was obtained with the mobile phase as Heptane:isopropyl alcohol in a ratio of 94:6 at absorbance 210 nm. In the reverse phase method Zorbax ODS 5 μm column was used. The mobile phase was water:methanol in a ratio 20:80 at an absorbance of 200 nm.

The isomers 12a and 12b thus separated were then reduced with diisobutyl aluminium hydride to yield either 13a or 13b respectively. The lactol 13a or 13b was further reacted at −15° C. to 10c preferably at −5° C. to 2° C. with the ylide obtained either from 4-carboxybutyltriphenyl-phosphonium bromide and sodium methylsulfinylmethide (obtained by reaction of sodium hydride and dimethyl sulphoxide) or from 4-carboxybutyltriphenyl-phosphonium bromide and sodium methylsulfinylmethide (obtained by reaction of sodium amide and dimethyl sulphoxide) to yield 9a or 9b. The esterification of 9a or 9b with methyl iodide and potassium carbonate gave (15R)-15-Methyl-PGF₂α Methyl ester (10a) or (15S)-15-Methyl-PGF₂α Methyl ester i.e. Carboprost methyl ester (10b). It is interesting to note that under the reaction conditions employed no epimerization of 10a to epimeric mixture of 10(RS) or 10b to an isomeric mixture of 10(RS) occurred. It was observed that in this case the chemical purification of 10a or 10b was much easy as the other side products or impurities were formed to a lesser extent.

### EXPERIMENTAL

All melting points reported are corrected. The HPLC instrument used for analysis is Shimadzu SPD-10A. For preparative work the instruments used were Agilent 1100 series supplied by Agilent Technologies and Hipersep LAB LC 50 supplied by Novasep. Optical rotations were recorded using Jasco DIP-370 digital polarimeter. ¹H or ¹³C NMR were recorded using a Bruker Avance DPX 200 NMR instrument. Anhydrous solvents were generally prepared by known procedure⁴.

(3aR,4R,5R,6aS)-5-triethylsilyloxy-4-[(1E,3S)-3-hydroxy-3-methyl oct-1-en-1-yl]hexahydro-2H-cyclopenta[b]furan-2-one, [12(RS)]. 1.011 Kg (2.646 mol) of Triethyl silyloxy PG enone (11) was dissolved in dry toluene or xylene (o, m, p or mixed). To this was slowly added 4.41 litres (13.23 mol) of methyl magnesium chloride in tetrahydrofuran (3.0 molar solution) under nitrogen atmosphere at −60° C. to −80° C. TLC monitored the progress of the reaction. On complete consumption of starting material the reaction mass is dumped in a saturated solution of ammonium chloride. The reaction mass was then filtered on hyflo bed. The organic layer was separated and the aqueous phase was extracted with ethyl acetate. The combined organic layers were washed with brine and concentrated to give oil.

Yield 1.032 Kg (98.47%). The ¹H and ¹³C NMR values matched with the reported values. [α]D −18° (c 2.62, methanol).

(3aR,4R,5R,6aS)-5-triethylsilyloxy-4-[(1E,3S)-3-hydroxy-3-methyl oct-1-en-1-yl]hexahydro-2H-cyclopenta[b]furan-2-one, [12(RS)] was also synthesized by following the same procedure mentioned above but instead using Dry THF as a solvent.

(3aR,4R,5R,6aS)-5-triethylsilyloxy-4-[(1E,3S)-3-hydroxy-3-methyl oct-1-en-1-yl]hexahydro-2H-cyclopenta[b]furan-2-ol, [13(RS)]. To a stirred solution of 775.0 gms (1.95 Moles) of (3aR,4R,5R,6aS)-5-triethylsilyloxy-4-[(1E,3S)-3-hydroxy-3-methyl oct-1-en-1-yl]hexahydro-2H-cyclopenta[b]furan-2-one, [12(RS)] in 9.0 litres of dry THF was added 4.566 litres (6.84 moles) of diisobutyl aluminium hydride (1.5M solution in toluene) at −78° C. The completion of the reaction was monitored by TLC. The reaction mixture was then dumped in ice-cold water. The reaction mass was then filtered on hyflo bed. The organic layer was separated and the aqueous phase was extracted with ethyl acetate. The combined organic layers were washed with brine and concentrated to give an oil. Yield 774.0 Gms (99.48%).

Methyl (Z)-7-(1R,2R,3R,5S)-3,5-dihydroxy-2-[(E)-(3RS)-3-hydroxy-3-methyl-1-octenyl)cyclopentyl]-5-heptenoate, [10(RS)]. Sodium hydride (60% dispersion in oil) 473.0 gms (11.82 moles) was treated with pet ether to make it free from oil dispersion. To this was added 4.73 litres of dimethyl sulphoxide and heated to 75° C. under nitrogen atmosphere. The resulting dark clear solution was cooled to ambient temperature and to this was added 2.588 Kg (5.83 moles) of 4-carboxybutyl triphenyl-phosphonium bromide, the resulting brick red coloured solution was cooled to −5° C. to 5° C. To this ylide, 774.0 gms (1.94 moles) of (3aR,4R,5R,6aS)-5-triethylsilyloxy-4-[(1E,3S)-3-hydroxy-3-methyl oct-1-en-1-yl]hexahydro-2H-cyclopenta[b]furan-2-ol, [13(RS)] was added and the solution stirred overnight at −2° C. The reaction was dumped in ice water and acidified with 10% solution of sodium bisulphate. After equilibration, the aqueous phase was extracted with ethyl acetate. The organic layers were combined and washed with brine. Concentration of this organic layer under vacuum gave crude product 9(RS). The product was not purified at this stage and taken up for esterification directly. The product was dissolved in acetone and to this was added 703.0 (5.0 moles) Gms of potassium carbonate followed by methyl iodide 636.0 ml (10.216 moles). The reaction mixture was stirred overnight. The reaction mixture is filtered through celite bed and is then dumped in water and extracted with ethyl acetate. The organic layer is then concentrated to give crude 10(RS). This crude material is then purified by column chromatography to yield chemically pure 10(RS).

Methyl (Z)-7-(1R,2R,3R,5S)-3,5-dihydroxy-2-[(E)-(3RS)-3-hydroxy-3-methyl-1-octenyl)cyclopentyl]-5-heptenoate, [10(RS)]. Sodium amide 245.0 gms (6.28 moles) was added to 2.5 litres of dimethyl sulphoxide followed by 1.4 Kg (3.16 moles) of 4-carboxybutyl triphenyl-phosphonium bromide, the resulting brick red solution was heated to 50° C. under nitrogen atmosphere for 1.0 to 2.0 hrs. The resulting dark clear solution was cooled to −5° C. to 5° C. To this ylide, 252.0 gms (0.633 moles) of (3aR,4R,5R,6aS)-5-triethylsilyloxy-4-[(1E,3S)-3-hydroxy-3-methyl oct-1-en-1-yl]hexahydro-2H-cyclopenta[b]furan-2-ol, [13(RS)] was added and the solution stirred overnight at −5° C. to 5° C. The reaction was dumped in ice water and acidified with 10% solution of sodium bisulphate. After equilibration, the aqueous phase was extracted with ethyl acetate. The organic layers were combined and washed with brine. Concentration of this organic layer under vacuum gave crude product. The product 9(RS) was not purified at this stage and taken up for esterification directly. The product was dissolved in acetone and to this was added 703.0 (5.0 moles) Gms of potassium carbonate followed by methyl iodide 636.0 ml (10.216 moles). The reaction mixture was stirred overnight. The reaction mixture is filtered through celite bed and is then dumped in water and extracted with ethyl acetate. The organic layer is then concentrated to give crude 10(RS). This crude material is then purified by column chromatography to give chemically pure 10(RS).

The synthesis of (15RS)-15-Methyl-PGF₂α methyl ester [10(RS)] was carried out in two ways. In the first method the Grignard reaction of Triethyl silyloxy PG enone (11) with methyl magnesium chloride was carried out in THF as a solvent. The product thus obtained was reduced with diisobutyl aluminium hydride and subsequently reacted with ylide formed using 4-carboxybutyl triphenyl-phosphonium bromide and sodium methylsulfinylmethide (obtained by reaction of sodium hydride or sodium amide and dimethyl sulphoxide). The free acid obtained, on esterification gave 10(RS) in 55% overall yield starting from Triethyl silyl PG enone (11) with a ratio of R:S being 40:60. In The second method Grignard reaction of Triethyl silyl PG enone (11) with methyl magnesium chloride was carried out in Xylene (o, m, p or mixed) or toluene as a solvent. The product thus obtained was reduced with diisobutyl aluminium hydride and subsequently reacted with ylide formed using 4-carboxybutyl triphenyl-phosphonium bromide and sodium methylsulfinylmethide (obtained by reaction of sodium hydride or sodium amide and dimethyl sulphoxide). The free acid obtained, on esterification gave 10(RS) in 75% overall yield starting from Triethyl silyl PG enone (11) with a ratio of R:S being 30:70. However in both the cases the 5-trans isomer was formed below 3%.

(3aR,4R,5R,6aS)-5-triethylsilyloxy-4-[1(E)-3-oxobut-1-en-1-yl]hexahydro-2H-cyclopenta[b]furan-2-one (15). 20.0 Gms (0.0956 moles) of (3aR,4R,5R,6aS)-5-Hydroxy-4-(E)-3-oxo-but-1-enyl-hexahydro-cyclopenta[b]furan-2-one (14) was added to 200 ml of dry pyridine. To this was added 26.0 gms (0.172 moles) of triethylchlorosilane and the reaction mixture heated at 60° C. The progress of the reaction was monitored by TLC. The reaction mixture was dumped in water and extracted with ethyl acetate. The organic layer was then washed with brine and concentrated to yield 30.3 gms (97.74%) of (15).

(3aR,4R,5R,6aS)-5-triethylsilyloxy-4-[(1E,3S)-3-hydroxy-3-methyl oct-1-en-1-yl]hexahydro-2H-cyclopenta[b]furan-2-one, [12(RS)]. 10 gms (0.030 moles) of (3aR,4R,5R,6aS)-5-triethylsilyloxy-4-[1(E)-3-oxobut-1-en-1-yl]hexahydro-2H cyclopenta[b]furan-2-one (15) was dissolved in 100 ml of toluene and cooled to −78° C. To this was added 77.0 ml (0.15 moles) of pentyl magnesium bromide (2.0 molar solution in diethyl ether) under nitrogen atmosphere. Completion of the reaction was monitored by TLC. The reaction was then quenched in a saturated solution of ammonium chloride and extracted with ethyl acetate. The organic layer was washed with brine and concentrated to yield 12(RS). The ¹H and ¹³C NMR values matched with the reported values. [α]_{D}−17° (c 2.57, methanol).

(3aR,4R,5R,6as)-5-triethylsilyloxy-4-[(1E,3S)-3-hydroxy-3-methyl oct-1-en-1-yl]hexahydro-2H-cyclopenta[b]furan-2-one, [12(RS)] obtained as a mixture of R:S isomer is separated into individual isomers using a preparative HPLC. The individual isomers 12a and 12b thus obtained are then reduced to 13a and 13b using diisobutyl aluminium hydride. Wittig reaction and esterification gave 10a and 10b respectively. The experimental conditions including molar proportions and solvents used for synthesizing 10a and 10b from 12a and 12b respectively (Scheme III) remain the same as that used to synthesize 10 as an RS mixture from 12(RS). The yield of Methyl (Z)-7-(1R,2R,3R,5S)-3,5-dihydroxy-2-[(E)-(3R)-3-hydroxy-3-methyl-1-octenyl)cyclopentyl]-5-heptenoate, (10a) and Methyl (Z)-7-(1R,2R,3R,5S)-3,5-dihydroxy-2-[(E)-(3S)-3-hydroxy-3-methyl-1-octenyl)cyclopentyl]-5-heptenoate (10b) obtained was 70-75% Starting from 12a and 12b respectively.

### REFERENCES

1. (a) S. Bergström, Science, 157,382 (1967); (b) “Prostaglandins” in Proceedings of Second Nobel Symposium, Stockholm, June, 1966, S. Bergström, and B Samuelsson, Ed., Almquist and Wiksell, Gebers Forlag A B, Stockholm, 1967.
2. (a) E. J Corey, N. M. Weinshenker, T. K. Schaaf, and W. Huber, J. Amer. Chem. Soc., 91, 5677 (1969). (b) E. J. Corey, T. K. Schaaf, W. Huber, U. Koelleker and N. M. Weinshenker, ibid., 92, 397 (1970). (c) E. J. Corey, S. M. Albonico, T. K. Schaaf, U. Koelleker and R. K. Varma, ibid., 93, 1491 (1971).
3. Practical HPLC method development—L. R. Synder, J. Kirkland and J. L. Glajch, John Wiley & sons Inc.
4. Vogel's Textbook of Practical Organic Chemistry, Fifth Edition. Longman Group UK Ltd. 1989.

1. A process for the preparation of carboprost methyl ester (10) 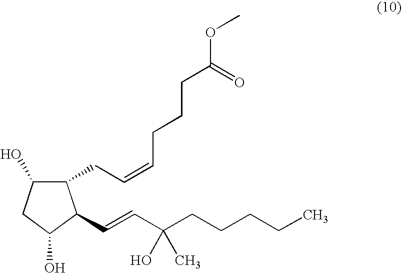
which process comprises the Wittig reaction of lactol (13) 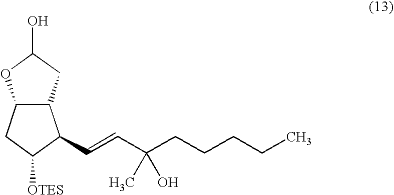
wherein OTES is a triethylsilyloxy group,
with an ylide of formula Ph₃P═CH—(CH₂)₃—COOH
at between −25° C. and +10° C. to give carboprost (9) 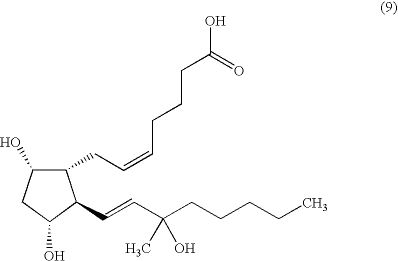
followed by esterification to give carboprost methyl ester 10.

2. A method as claimed in claim 1 wherein the ylide of formula Ph₃P═CH—(CH₂)₃—COOH is formed by the reaction of 4-carboxybutyltriphenyl-phosphonium bromide and sodium methylsulfinylmethide.

3. A method as claimed in claim 2 wherein sodium methylsulfinylmethide is prepared by the reaction of sodium amide and dimethyl sulphoxide.

4. A process as claimed in claim 1 wherein carboprost methyl ester 10(RS) is produced and then separated into carboprost methyl esters 10R and 10S

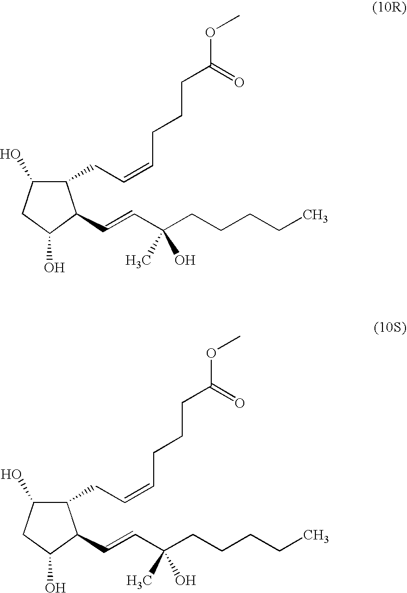

5. A process as claimed in claim 1 wherein the lactol 13 is prepared by reduction of (3aR,4R,5R,6aS)-5-triethylsilyloxy-4-[(1E,3S)-3-hydroxy-3-methyl oct-1-en-1-yl]hexahydro-2H-cyclopenta[b]furan-2-one (12)

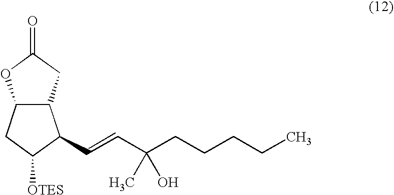

6. A process as claimed in claim 5 wherein (3aR,4R,5R,6aS)-5-triethylsilyloxy-4-[(1E,3S)-3-hydroxy-3-methyl oct-1-en-1-yl]hexahydro-2H-cyclopenta[b]furan-2-one (12) is dissolved in a solvent selected from tetrahydrofuran, toluene or xylene, and about 3.5 molar equivalents of diisobutylaluminium hydride are used at between −60° C. and −78° C.

7. A process as claimed in claim 6 (3aR,4R,5R,6aS)-5-triethylsilyloxy-4-[(1E,3S)-3-hydroxy-3-methyl oct-1-en-1-yl]hexahydro-2H-cyclopenta[b]furan-2-one (12) is prepared by Grignard reaction of triethylsilyloxy PG enone (11) 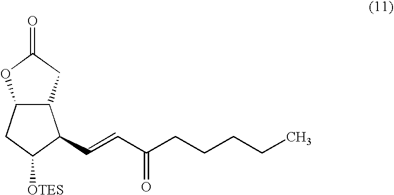
dissolved in a solvent selected from tetrahydrofuran, xylene or toluene.

8. A process as claimed in claim 7 wherein the ratio of triethylsilyloxy PG enone (11) to Grignard reagent is about 1:5 moles.

9. A process as claimed in claim 5 wherein (3aR,4R,5R,6aS)-5-triethylsilyloxy-4-[(1E,3S)-3-hydroxy-3-methyl oct-1-en-1-yl]hexahydro-2H-cyclopenta[b]furan-2-one (12) is prepared by Grignard reaction with compound 15

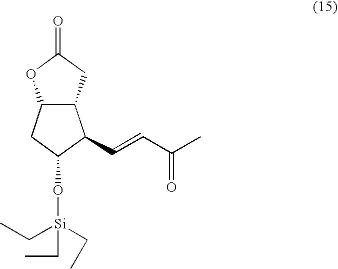

10. A process as claimed in claim 9 wherein compound 15 is prepared by protection of the hydroxyl group of compound 14 with triethyl silyl chloride (triethylchlorosilane).

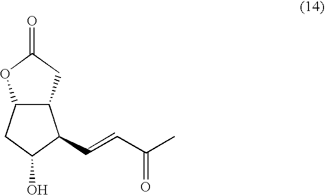

11. A process as claimed in claim 1 wherein either the R or S isomer of compound 13 is used in the process.

12. A process as claimed in claim 5 wherein either the R or S isomer of compound 12 is used in the process.

13. A process as claimed in claim 12 wherein compound 12 is firstly separated into individual isomers and either the R or S isomer is used in the process.

14. A process according to claim 1 wherein any separation into individual isomers is carried out using preparative scale HPLC.

15. A process according to claim 1 for the preparation of carboprost methyl ester 10R.

16. A process according to claim 1 for the preparation of carboprost methyl ester 10S.

17. A process according to claim 1 for the preparation of carboprost methyl ester 10RS.
